(19) United States
(12) Patent Application Publication
(10) Pub. No.: US 2024/0285913 A1
(43) Pub. Date: Aug. 29, 2024

(54) CATHETER CAPABL E OF PREVENTING GUIDE WIRE RETENTION AND DETACHABLE MAGNETIC ATTRACTION MECHANISM THEREOF

(71) Applicant: Chang-Shan CHEN, Taipei City (TW)

(72) Inventors: Chang-Shan CHEN, Taipei City (TW); Chien-Ya HSU, Taipei City (TW); Yen-Lin HUANG, Taipei City (TW); Wei-Yi LIU, Taipei City (TW)

(21) Appl. No.: 18/505,724

(22) Filed: Nov. 9, 2023

(30) Foreign Application Priority Data
Feb. 23, 2023 (TW) 112106762

(51) Int. Cl. A61M 25/09

(52) U.S. Cl. CPC A61M 25/09041; A61M 2025/09125; A61M 2025/09133; A61M 2205/0288

(57) ABSTRACT

The present invention provides a catheter capable of preventing guide wire retention comprising a catheter body and a guide wire. The guide wire is made of a magnetic material or provided with a magnetic element and is configured to be inserted through the catheter body; wherein the catheter body is provided with at least one detachable magnetic attraction mechanism for attracting the guide wire and thereby holding the guide wire in position after the guide wire is inserted through the catheter body.

### BACKGROUND OF THE INVENTION

### 1. Technical Field

The present invention relates to a catheter capable of preventing guide wire retention and a detachable magnetic attraction mechanism thereof. More particularly, the invention relates to a catheter that uses a detachable magnetic attraction mechanism to stabilize a guide wire and prevent guide wire retention, and to the detachable magnetic attraction mechanism of the catheter.

### 2. Description of Related Art

When placing an indwelling catheter such as a central venous catheter (CVC), a peripheral intravenous central catheter (PICC), a double-lumen catheter, or a pigtail catheter in a clinical setting, the metal guide wire used in the catheter placing procedure may, under emergency circumstances or due to human error, be retained in the patient's body and thus cause serious injury to the patient.

Each year in the United States, about five million CVCs are placed into patients in the perioperative period. While strategies for effectively preventing infectious and mechanical complications associated with CVC placement have been established in recent years, guide wire retention remains a problem without an effective solution. The incidence of guide wire retention is difficult to track, but a survey has estimated that the incidence of guide wire retention-related complications may be one case in every three thousand catheter insertions.

An analysis of closed claims data reveals that the percentage of CVC-related claims has been increasing in a statistically significant manner, and that 25 cases of the claims are attributable to guide wire retention, with one case leading to death and another case resulting in permanent disability. Such incidents are usually caused by an operator's inexperience, fatigue, or lack of concentration, or by inadequate supervision of trainees. Guide wire retention poses a major risk to a patient's surgical safety, but neither the academia nor relevant practitioners have come up with a concrete preventive strategy. It is therefore imperative to provide improvement measures for addressing the issue of guide wire retention.

### BRIEF SUMMARY OF THE INVENTION

One objective of the present invention is to provide a catheter capable of preventing guide wire retention. The catheter capable of preventing guide wire retention comprises a catheter body and a guide wire. The guide wire is made of a magnetic material or provided with a magnetic element and is configured to be inserted through the catheter body. The catheter body is provided with at least one detachable magnetic attraction mechanism for attracting the guide wire and thereby holding the guide wire in position after the guide wire is inserted through the catheter body.

According to one embodiment of the present invention, the catheter body comprises a hub, a puncture needle provided on a side of the hub, and an extension tube connected to an opposite side of the hub; the catheter body further comprises a guide channel; the guide channel extends through the extension tube, the hub, and the puncture needle; and the guide wire is inserted into the puncture needle and then passed through the extension tube via the guide channel.

According to one embodiment of the present invention, the detachable magnetic attraction mechanism comprises a magnetic positioning member configured to be provided on an adapter of the extension tube, the magnetic positioning member comprises a base and a magnetically attractive element, the base is provided with a through hole, the base is further provided with a locking portion corresponding to an end of the through hole, the locking portion is configured to be locked to the adapter, and the magnetically attractive element is provided at the base.

According to one embodiment of the present invention, the detachable magnetic attraction mechanism comprises a magnetic suspension mechanism configured to be provided on the extension tube, and the magnetic suspension mechanism comprises a tie connected to the extension tube and a magnetically attractive element provided on the tie.

According to one embodiment of the present invention, the detachable magnetic attraction mechanism comprises a magnetic slide block provided on a tube body of the extension tube, the magnetic slide block comprises a slide base and a magnetically attractive element provided in the slide base, and the slide base comprises a positioning hole configured to allow passage of the extension tube.

According to one embodiment of the present invention, the detachable magnetic attraction mechanism comprises a magnetic slide clip provided on a tube body of the extension tube, the magnetic slide clip comprises a clip element and a magnetically attractive element provided in the clip element, and the clip element comprises a positioning groove for receiving the extension tube when the clip element is clipped to the extension tube.

One objective of the present invention is to provide a detachable magnetic attraction mechanism for stabilizing a guide wire, configured to be provided in relation to a catheter, the detachable magnetic attraction mechanism comprises a magnetic positioning member. The magnetic positioning member comprises a base and a magnetically attractive element. The base is provided with a through hole, the base is further provided with a locking portion corresponding to an end of the through hole, and the locking portion is configured to be locked to an opening at an end of the catheter. The magnetically attractive element is provided at the base and is configured to attract the guide wire after the guide wire is inserted through the catheter.

One objective of the present invention is to provide a detachable magnetic attraction mechanism for stabilizing a guide wire, configured to be provided in relation to a catheter, the detachable magnetic attraction mechanism comprises a magnetic suspension mechanism. The magnetic suspension mechanism comprises a tie and a magnetically attractive element. The tie is connected to the catheter. The magnetically attractive element is provided on the tie and is configured to attract the guide wire after the guide wire is inserted through the catheter, thereby exposing the guide wire from an opening at an end of the catheter.

One objective of the present invention is to provide a detachable magnetic attraction mechanism for stabilizing a guide wire, configured to be provided in relation to a catheter, the detachable magnetic attraction mechanism comprises a magnetic slide block. The magnetic slide block comprises a slide base and a magnetically attractive element. The slide base comprises a positioning hole, wherein the positioning hole allows passage of the catheter. The magnetically attractive element is provided in the slide base and is configured to attract the guide wire after the guide wire is inserted through the catheter.

One objective of the present invention is to provide a detachable magnetic attraction mechanism for stabilizing a guide wire configured to be provided in relation to a catheter, the detachable magnetic attraction mechanism comprises a magnetic slide clip. The magnetic slide clip comprises a clip element and a magnetically attractive element. The clip element comprises a positioning groove, wherein the positioning groove is configured to receive the catheter when the clip element is clipped to the catheter. The magnetically attractive element is provided in the clip element and is configured to attract the guide wire after the guide wire is inserted through the catheter.

The present invention uses a detachable magnetic attraction mechanism to stabilize a guide wire, to ensure that the guide wire is exposed from one end of the catheter guided by the guide wire, and to thereby reduce the chance that the operator may forget to take out the guide wire. Moreover, the guide wire will be magnetically attracted to the detachable magnetic attraction mechanism and hence kept from sliding into a blood vessel inadvertently. The invention, therefore, can lower the risk of guide wire retention.

### BRIEF DESCRIPTION OF THE SEVERAL VIEWS OF THE DRAWINGS

FIG. 1 is schematic drawings of a catheter capable of preventing guide wire retention according to the present invention;

FIG. 2 shows the exterior of the first embodiment of the detachable magnetic attraction mechanism of the invention;

FIG. 3 shows the exterior of the second embodiment of the detachable magnetic attraction mechanism of the invention;

FIG. 4 shows the exterior of the third embodiment of the detachable magnetic attraction mechanism of the invention;

FIG. 5 shows the exterior of the fourth embodiment of the detachable magnetic attraction mechanism of the invention;

FIG. 6 shows the exterior of the first embodiment of the catheter capable of preventing guide wire retention according to the invention;

FIG. 7 is a partial enlarged view of the first embodiment of the catheter capable of preventing guide wire retention according to the invention;

FIG. 8 shows various shapes and configurations of the magnetic positioning member in the first embodiment of the catheter capable of preventing guide wire retention according to the invention;

FIG. 9 shows the exterior of the second embodiment of the catheter capable of preventing guide wire retention according to the invention;

FIG. 10 is a partial enlarged view of the second embodiment of the catheter capable of preventing guide wire retention according to the invention;

FIG. 11 shows various shapes and configurations of the magnetic suspension mechanism in the second embodiment of the catheter capable of preventing guide wire retention according to the invention;

FIG. 12 shows the exterior of the third embodiment of the catheter capable of preventing guide wire retention according to the invention;

FIG. 13 is a partial enlarged view of the third embodiment of the catheter capable of preventing guide wire retention according to the invention;

FIG. 14 shows various shapes and configurations of the magnetic slide block in the third embodiment of the catheter capable of preventing guide wire retention according to the invention;

FIG. 15 shows the exterior of the fourth embodiment of the catheter capable of preventing guide wire retention according to the invention;

FIG. 16 is a partial enlarged view of the fourth embodiment of the catheter capable of preventing guide wire retention according to the invention;

FIG. 17 shows various shapes and configurations of the magnetic slide clip in the fourth embodiment of the catheter capable of preventing guide wire retention according to the invention;

FIG. 18 shows the exterior of the fifth embodiment of the catheter capable of preventing guide wire retention according to the invention;

FIG. 19 shows a state of use of a different embodiment of the guide wire in the invention; and

FIG. 20 shows the process flow of guiding a central venous catheter according to the invention into a vein.

### DETAILED DESCRIPTION OF THE INVENTION

According to the usual working methods, the various features and components in the figures are not drawn according to the actual scale. The drawings present the specific features and components related to the present invention in the best way. In addition, between different drawings, the same or similar element symbols refer to similar elements and components.

The following embodiments should not be considered as excessively limiting the present invention. Those with ordinary knowledge in the technical field to which the present invention belongs can modify and change the embodiments discussed herein without departing from the spirit or scope of the present invention, and the variation and modification may still fall into the scope of the present invention.

As used herein, terms such as “comprise,”“include,”“have,” and “contain” are inclusive or open-ended unless otherwise stated and therefore do not exclude elements or steps that are not specified. The terms “a” and “said” may be construed as referring to a single referent or plural referents. The term “one or a plurality of” means “at least one” and therefore may be used to identify a single feature or a mixture/combination of features. Moreover, unless otherwise stated, the term “provided on an article” as used in this specification and the appended claims may be construed as being directly or indirectly attached to a surface of the article or as contacting the surface of the article in other ways, wherein the definition of the surface should be determined according to the context and common knowledge in the art.

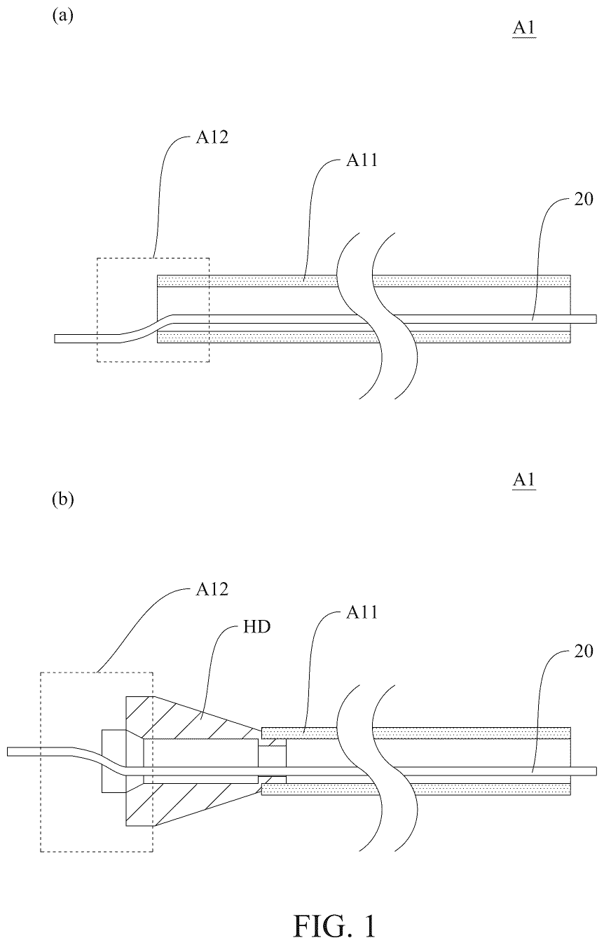

Please refer to FIG. 1 for schematic drawings of a catheter capable of preventing guide wire retention according to the present invention. As shown in the drawing, the invention provides a catheter A1 capable of preventing guide wire retention, and the catheter A1 includes a catheter body A11 and a guide wire 20. The guide wire 20 is made of a magnetic material or is provided with a magnetic element. The guide wire 20 is configured to extend through the catheter body A11. The catheter body A11 is provided with at least one detachable magnetic attraction mechanism A12 for attracting the guide wire 20 and thereby holding the guide wire 20 in position after the guide wire 20 is inserted through the catheter body A11. The catheter body A11 may be, for example but not limited to, a central venous catheter (CVC), a peripheral intravenous central catheter (PICC), a double-lumen catheter, a pigtail catheter, or other types of catheters whose placement requires the assistance of the guide wire 20; the invention has no limitation in this regard.

In the embodiment shown in FIG. 1(a), the detachable magnetic attraction mechanism A12 is provided directly on the catheter body A11. In the embodiment shown in FIG. 1(b), the detachable magnetic attraction mechanism A12 is provided on an adapter HD connected to the catheter body A11. The present invention has no limitation on whether the detachable magnetic attraction mechanism A12 is provided directly or indirectly on the catheter body A11.

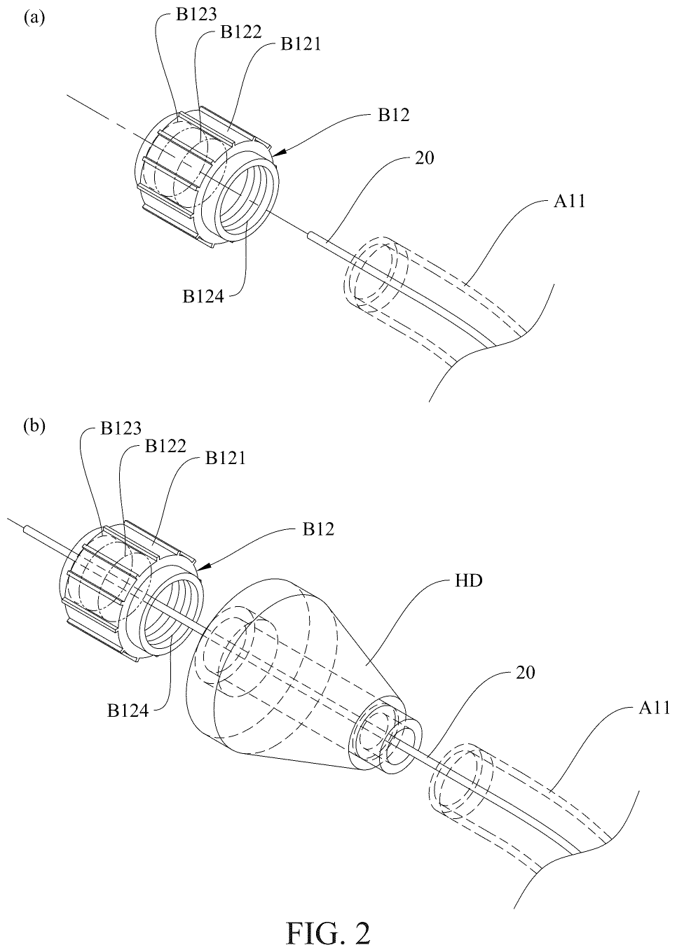

Various embodiments of the detachable magnetic attraction mechanism are described below. To begin with, please refer to FIG. 2, which shows the exterior of the first embodiment of the detachable magnetic attraction mechanism of the present invention. As shown in the drawing, the detachable magnetic attraction mechanism according to this embodiment includes a magnetic positioning member B12. The magnetic positioning member B12 includes a base B121 and a magnetically attractive element B122. The base B121 is provided with a through hole B123. The base B121 is further provided with a locking portion B124 corresponding to one end of the through hole B123. The locking portion B124 is configured to be locked to the opening at one end of the catheter body A11. The magnetically attractive element B122 may be provided at any position on the base B121 and is configured to attract the guide wire 20 after the guide wire 20 is inserted through the catheter body A11. The detachable magnetic attraction mechanism can be designed to be provided at an opening of a catheter body A11 of any diameter, in order to use the magnetically attractive element B122 to attract the guide wire 20 after the guide wire 20 is inserted through the catheter body A11.

In one embodiment, the locking portion B124 may be, for example but not limited to, a screw thread structure, a position-limiting rib, an elastic clip, an elastic gripper, or other similar mechanisms provided on the inner wall of the through hole B123; the present invention has no limitation in this regard.

In the embodiment shown in FIG. 2(a), the magnetic positioning member B12 is provided directly on the catheter body A11. In the embodiment shown in FIG. 2(b), the magnetic positioning member B12 is provided on the adapter HD of the catheter body A11. The present invention has no limitation on whether the magnetic positioning member B12 is provided directly or indirectly on the catheter body A11.

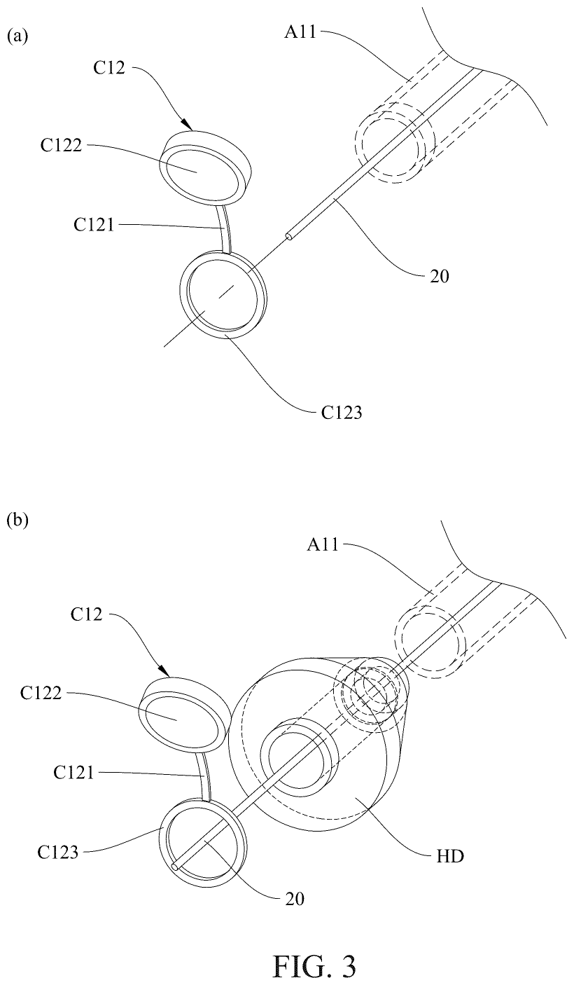

Please refer to FIG. 3, which shows the exterior of the second embodiment of the detachable magnetic attraction mechanism of the present invention. As shown in the drawing, the detachable magnetic attraction mechanism according to this embodiment includes a magnetic suspension mechanism C12. The magnetic suspension mechanism C12 includes a tie C121 and a magnetically attractive element C122 provided on the tie C121. The tie C121 is connected to one end of the catheter body A11. The magnetically attractive element C122 is configured to attract the guide wire 20 such that after being inserted through the catheter body A11, the guide wire 20 is exposed from the opening at the end of the catheter body A11.

In one embodiment, the tie C121 may be provided with a mounting ring C123 configured to be mounted around and fixed on the catheter body A11. In another embodiment, the tie C121 may be integrally formed with the catheter body A11. The present invention has no limitation on how the tie C121 and the catheter body A11 are joined to each other.

In one embodiment, the tie C121 may be directly joined (e.g., adhesively bonded, fastened, or locked) to the magnetically attractive element C122, indirectly joined (e.g., adhesively bonded, fastened, or locked) to the magnetically attractive element C122 (e.g., with the tie fixed to or integrally formed with a base on which a magnet is fixed), or coupled to the magnetically attractive element C122 in other similar ways; the present invention has no limitation in this regard.

In one embodiment, the tie C121 may be, for example but not limited to, a pliable material, an elastic material, or a plastic material. The use of such a material makes it easier to adjust the position of the magnetically attractive element C122.

In the embodiment shown in FIG. 3(a), the magnetic suspension mechanism C12 is provided directly on the catheter body A11. In the embodiment shown in FIG. 3(b), the magnetic suspension mechanism C12 is provided on the adapter HD of the catheter body A11. The present invention has no limitation on whether the magnetic suspension mechanism C12 is provided directly or indirectly on the catheter body A11.

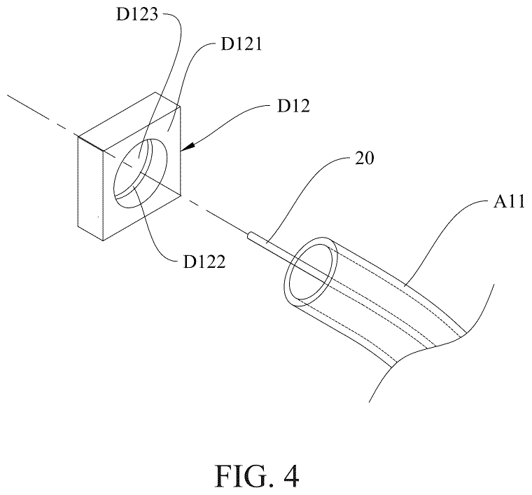

Please refer to FIG. 4, which shows the exterior of the fourth embodiment of the detachable magnetic attraction mechanism of the present invention. As shown in the drawing, the detachable magnetic attraction mechanism according to this embodiment includes a magnetic slide block D12. The magnetic slide block D12 includes a slide base D121 and a magnetically attractive element D122 provided in the slide base D121. The slide base D121 includes a positioning hole D123 through which the catheter body A11 can extend. The magnetically attractive element D122 is provided in the slide base D121 and is configured to attract the guide wire 20 after the guide wire 20 is inserted through the catheter body A11.

In one embodiment, the positioning hole D123 in the slide base D121 may have a diameter slightly smaller than the outer diameter of the catheter body A11 in order to increase the resistance the slide base D121 will encounter when moved on the catheter body A11. In another embodiment, the inner wall of the positioning hole D123 may have an interfering structure or microstructures or have received a certain surface treatment in order to increase the resistance the slide base D121 will encounter when moved on the catheter body A11. The present invention has no limitation on how the positioning hole D123 produces such resistance.

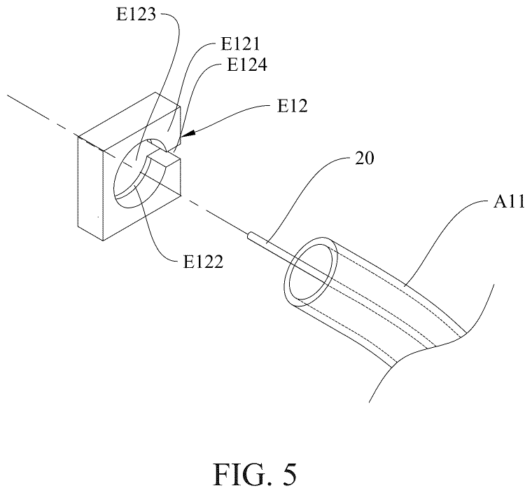

Please refer to FIG. 5, which shows the exterior of the fourth embodiment of the detachable magnetic attraction mechanism of the present invention. As shown in the drawing, the detachable magnetic attraction mechanism according to this embodiment includes a magnetic slide clip E12. The magnetic slide clip E12 includes a clip element E121 and a magnetically attractive element E122. The clip element E121 includes a positioning groove E123 for receiving the catheter body A11 to which the clip element E121 is clipped. The magnetically attractive element E122 is provided in the clip element E121 and is configured to attract the guide wire 20 after the guide wire 20 is inserted through the catheter body A11.

In one embodiment, the positioning groove E123 in the clip element E121 may have a diameter slightly smaller than the outer diameter of the catheter body A11 in order to increase the resistance the clip element E121 will encounter when moved on the catheter body A11. In another embodiment, the inner wall of the positioning groove E123 may have an interfering structure or microstructures or have received a certain surface treatment in order to increase the resistance the clip element E121 will encounter when moved on the catheter body A11. The present invention has no limitation on how the positioning groove E123 produces such resistance. One lateral side of the clip element E121 has a lateral groove E124 in communication with the positioning groove E123 so that the catheter body A11 can be fitted into the positioning groove E123 through the lateral groove E124 in the lateral side of the clip element E121.

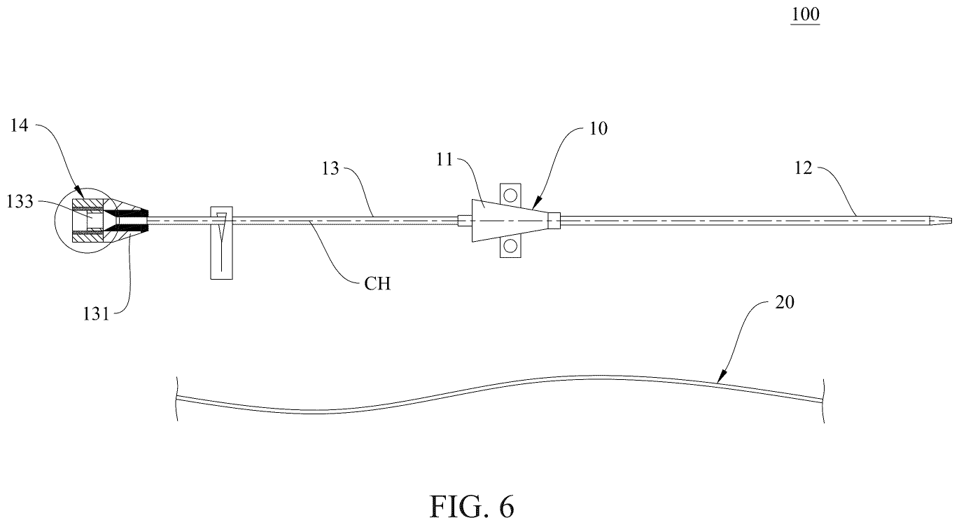

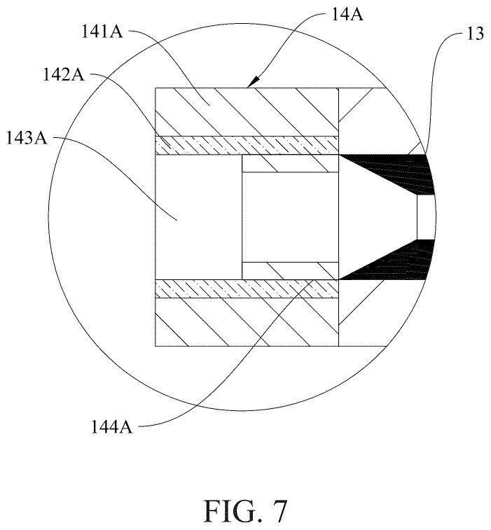

The first embodiment of the catheter capable of preventing guide wire retention according to the present invention is described below. While the description of this embodiment makes reference to a central venous catheter (CVC), the invention is applicable to not only a CVC, but also a peripheral intravenous central catheter (PICC), a double-lumen catheter, a pigtail catheter, and other catheters whose placement requires the assistance of the guide wire 20. There is no limitation on the type of the catheter to which the invention is applied. Please refer to FIG. 6 and FIG. 7, which respectively show the exterior and a partial enlarged view of the first embodiment of the catheter capable of preventing guide wire retention according to the invention.

This embodiment provides a catheter 100 capable of preventing guide wire retention. The catheter 100 includes a catheter body 10 and a guide wire 20. The catheter body 10 includes a hub 11, a puncture needle 12 provided on one side of the hub 11, and an extension tube 13 connected to the opposite side of the hub 11. The catheter body 10 includes a guide channel CH that extends through the extension tube 13, the hub 11, and the puncture needle 12. The guide wire 20 is made of a magnetic material and is configured to enter the guide channel CH through an opening 133 of the extension tube 13 and then pass through the puncture needle 12. The extension tube 13 is provided with a detachable magnetic attraction mechanism 14 for attracting the guide wire 20 and thereby holding the guide wire 20 in position after the guide wire 20 is inserted through the guide channel CH.

In one embodiment, the term “magnetic material” refers to a substance that can be attracted to a magnetically attractive material. More specifically, a magnetic material may be, for example, a substance that is composed of a transition element (e.g., iron, cobalt, or nickel) or an alloy thereof and that produces magnetism directly or indirectly; the present invention has no limitation in this regard.

In one embodiment, the term “magnetic attraction mechanism” refers to an object or device that can produce a magnetic force by itself, such as a magnet or a lodestone; the present invention has no limitation in this regard. The detachable magnetic attraction mechanism 14 serves as a magnetic dipole element, can attract a ferromagnetic substance such as iron, nickel, cobalt, or other similar metals, and can therefore attract the guide wire 20. The term “detachable” refers to the magnetic attraction mechanism 14 being removable in normal use without causing damage to the catheter body 10 or the magnetic attraction mechanism 14 itself, the objective being to prevent the magnetic attraction mechanism 14 from affecting the postoperative life and other medical treatments of a patient in whom a catheter has been placed with the assistance of the magnetic attraction mechanism 14, and to reuse the magnetic attraction mechanism 14 removed, if possible.

In one embodiment, the term “guide channel” refers to a channel composed of three parts, namely the channel in the extension tube 13, the channel in the hub 11, and the channel in the catheter body 10. The guide channel CH may or may not be provided therein with a one-way valve for preventing reverse flow of blood. Such a one-way valve may be provided, for example, between the extension tube 13 and the hub 11 and will not hinder passage of the guide wire 20.

In one embodiment, the puncture needle 12 may be, for example, any indwelling needle that is biocompatible. More specifically, the puncture needle 12 may be made of a plastic material for example; the present invention has no limitation in this regard.

In this embodiment, the detachable magnetic attraction mechanism 14 includes a magnetic positioning member 14A configured to be provided on an adapter 131 connected to the extension tube 13. In one embodiment, the magnetic positioning member 14A includes a base 141A and a magnetically attractive element 142A. The base 141A is provided with a through hole 143A. The base 141A is further provided with a locking portion 144A corresponding to one end of the through hole 143A. The locking portion 144A is configured to be locked to the adapter 131. The magnetically attractive element 142A may be provided at any position on the base 141A. In one embodiment, the magnetically attractive element 142A may be provided on the inner wall of the through hole 143A of the base 141A. In another embodiment, the magnetically attractive element 142A may be provided on the outer side of the base 141A. In yet another embodiment, the magnetically attractive element 142A may be embedded in the inner wall of the base 141A. Changes in position of the magnetically attractive element 142A shall be viewed as falling within the scope of the present invention.

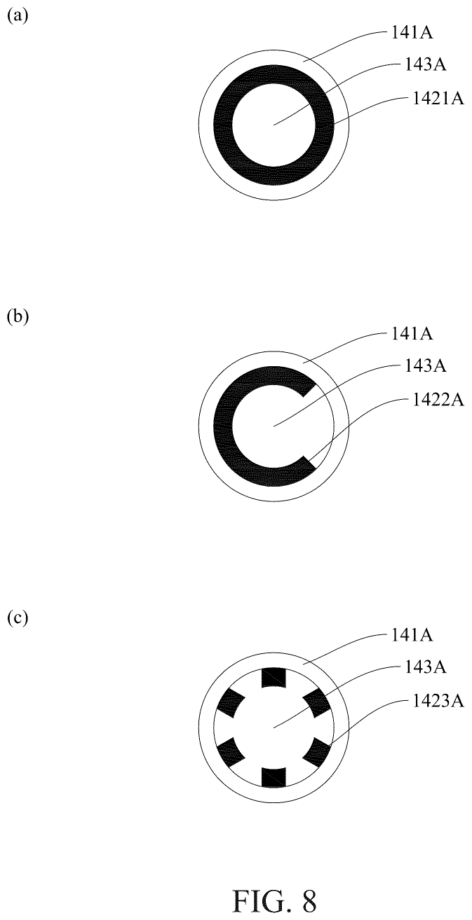

For the shape and configuration of the magnetically attractive element 142A of the magnetic positioning member 14A, please refer to FIG. 8, which shows various shapes and configurations of the magnetic positioning member in the first embodiment of the catheter capable of preventing guide wire retention according to the present invention. In the embodiment shown in FIG. 8(a), the magnetically attractive element 1421A is provided on, and extends along, the entire inner periphery of the base 141A and has an O shape by way of example. In the embodiment shown in FIG. 8(b), the magnetically attractive element 1422A is provided on, and extends along, a portion of the inner periphery of the base 141A and has a C shape by way of example. In the embodiment shown in FIG. 8(c), the magnetic attractive member 1423A is provided on, and extends at intervals along, the inner periphery of the base 141A. It should be noted that while FIG. 6 to FIG. 8 show the magnetically attractive element 1422A as provided on the inner wall of the through hole 143A of the base 141A, the magnetically attractive element 1422A may instead be provided on the outer side of the base 141A or be embedded directly in the inner wall of the base 141A; the invention has no limitation in this regard.

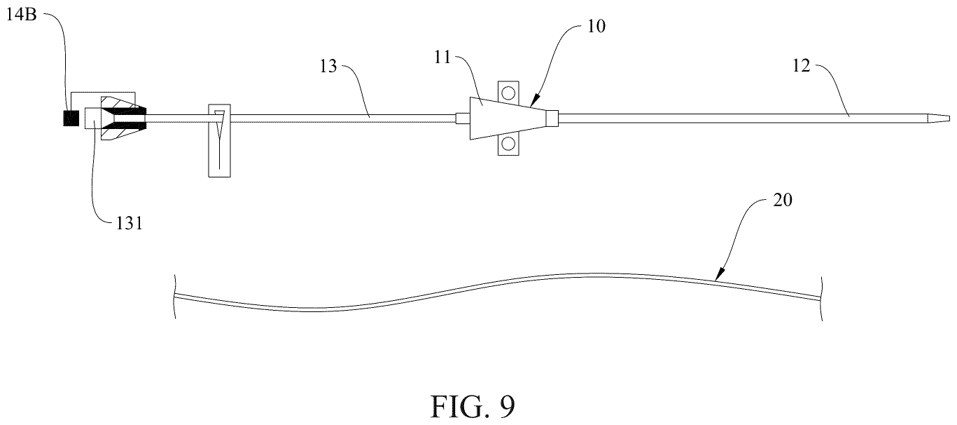

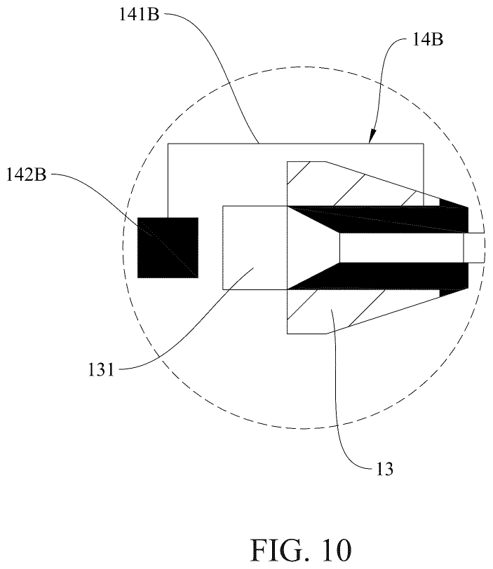

The second embodiment of the catheter capable of preventing guide wire retention according to the present invention is described below. Please refer to FIG. 9 and FIG. 10, which respectively show the exterior and a partial enlarged view of the second embodiment of the catheter capable of preventing guide wire retention according to the invention.

This embodiment is different from the first embodiment only in the design of the detachable magnetic attraction mechanism; all the remaining parts of this embodiment are identical to their respective counterparts in the first embodiment and therefore will not be described repeatedly. In this embodiment, the detachable magnetic attraction mechanism includes a magnetic suspension mechanism 14B configured to be provided on the extension tube 13. The magnetic suspension mechanism 14B includes a tie 141B connected to the extension tube 13 and a magnetically attractive element 142B provided on the tie 141B. For example, the tie 141B may be integrally formed with the extension tube 13 or positioned on the extension tube 13 via a positioning mechanism (e.g., a mounting ring or clamping plates); the present invention has no limitation in this regard.

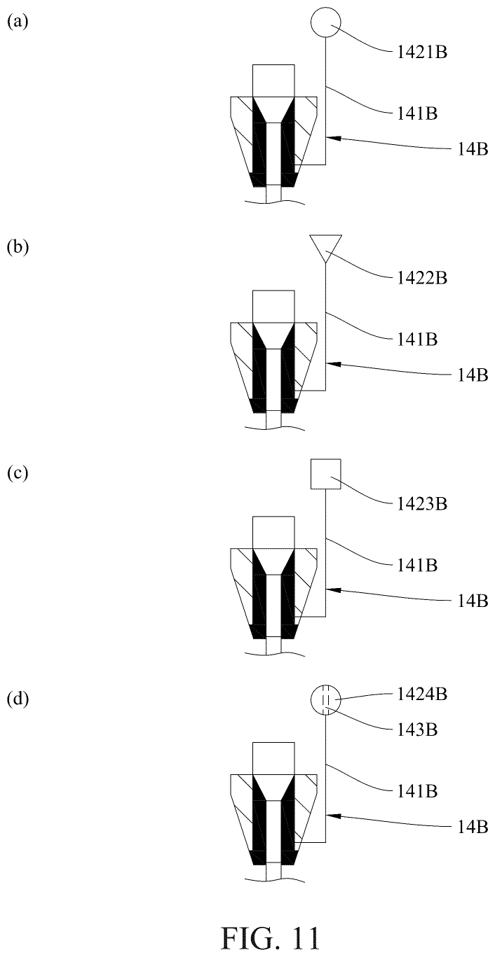

The shape of the magnetic suspension mechanism 14B may be, for example but not limited to, any of the following. Please refer to FIG. 11, which shows various shapes and configurations of the magnetic suspension mechanism in the second embodiment of the catheter capable of preventing guide wire retention according to the present invention. In the embodiment shown in FIG. 11(a), the magnetically attractive element 1421B of the magnetic suspension mechanism 14B is circular by way of example. In the embodiment shown in FIG. 11(b), the magnetically attractive element 1422B of the magnetic suspension mechanism 14B is triangular by way of example. In the embodiment shown in FIG. 11(c), the magnetically attractive element 1423B of the magnetic suspension mechanism 14B is square by way of example. In the embodiment shown in FIG. 11(d), the magnetically attractive element 1424B of the magnetic suspension mechanism 14B may have any shape, and the magnetically attractive element 1424B is provided with a positioning groove 143B in which the guide wire 20 can be fitted so as to be securely positioned. Changes in configuration of the magnetic suspension mechanism 14B shall be viewed as falling within the scope of the invention.

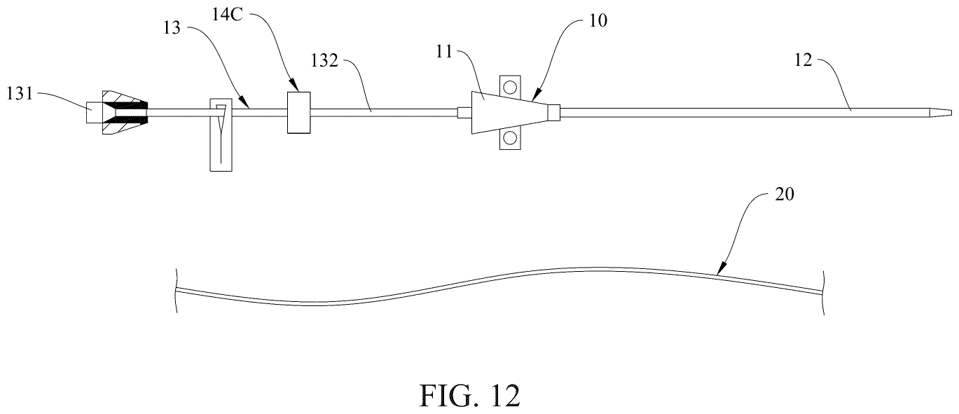

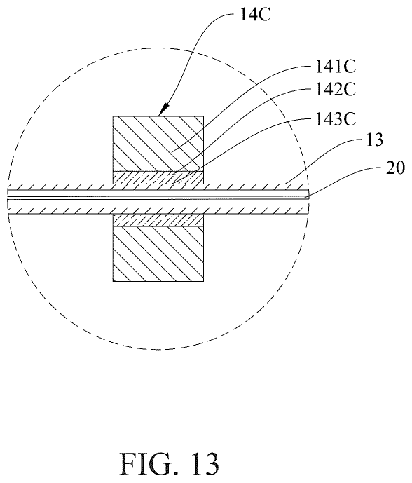

The third embodiment of the catheter capable of preventing guide wire retention according to the present invention is described below. Please refer to FIG. 12 and FIG. 13, which respectively show the exterior and a partial enlarged view of the third embodiment of the catheter capable of preventing guide wire retention according to the invention.

This embodiment is different from the first embodiment only in the design of the detachable magnetic attraction mechanism too; all the remaining parts of this embodiment are identical to their respective counterparts in the first embodiment and therefore will not be described repeatedly. In this embodiment, the detachable magnetic attraction mechanism includes a magnetic slide block 14C provided on the tube body 132 of the extension tube 13. The magnetic slide block 14C includes a slide base 141C and a magnetically attractive element 142C provided in the slide base 141C. The slide base 141C includes a positioning hole 143C through which the extension tube 13 can extend to allow movement of the magnetic slide block 14C on and along the extension tube 13. In one embodiment, the slide base 141C and the magnetically attractive element 142C of the magnetic slide block 14C may jointly form a single magnet, or the slide base 141C may be implemented as a magnetic or non-magnetic element; all such changes shall be viewed as falling within the scope of the present invention. The positioning hole 143C of the magnetic slide block 14C is designed according to the exterior shape of the extension tube 13, and the diameter of the positioning hole 143C is slightly smaller than the outer diameter of the extension tube 13 in order to provide a small amount of interference and thereby position the magnetic slide block 14C on the extension tube 13. The positioning hole 143C of the magnetic slide block 14C should be sized not to hinder the flow of liquid in the extension tube 13, but to provide a suitable clearance between the magnetic slide block 14C and the extension tube 13 so that the magnetic slide block 14C can be pushed along the extension tube 13. The magnetic slide block 14C is so designed that, once entering the extension tube 13, the guide wire 20 can be pushed out of the extension tube 13 through magnetic attraction by the magnetic slide block 14C. It should be noted that if the guide wire 20 is magnetic throughout, any portion of the guide wire 20 will be able to interact with the magnetic slide block 14C, making it difficult for the magnetic slide block 14C to push the guide wire 20 out of the extension tube 13. As a solution, the guide wire 20 may be rendered magnetic either partially or by attaching an additional magnetic element to the guide wire 20.

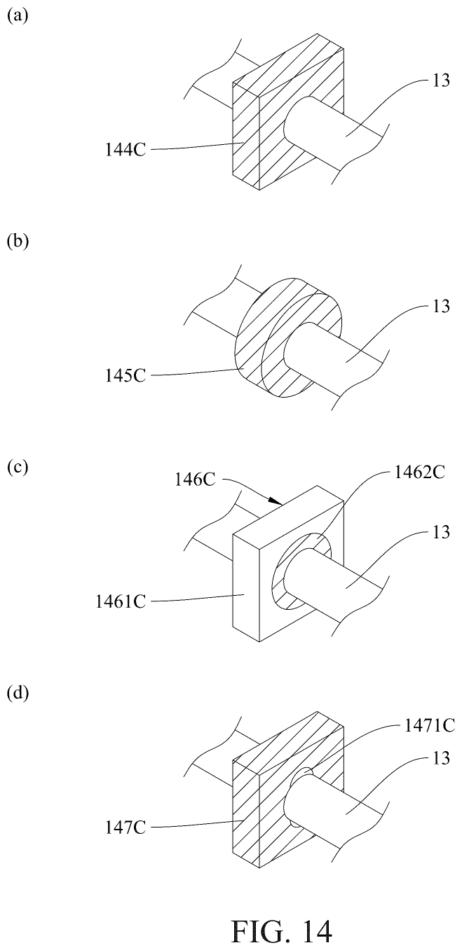

The shape of the magnetic slide block 14C may be, for example but not limited to, any of the following. Please refer to FIG. 14, which shows various shapes and configurations of the magnetic slide block in the third embodiment of the catheter capable of preventing guide wire retention according to the present invention. In the embodiment shown in FIG. 14(a), the magnetic slide block 144C may have any shape, such as a square shape. In the embodiment shown in FIG. 14(b), the magnetic slide block 145C may have any shape, such as a circular shape. In the embodiment shown in FIG. 14(c), the slide base 1461C of the magnetic slide block 146C may be a non-magnetic housing of any shape, and the magnetically attractive element 1462C in the magnetic slide block 146C may have any shape, such as a circular shape. In the embodiment shown in FIG. 14(d), the magnetic slide block 147C may have any shape, and the positioning hole 1471C of the magnetic slide block 147C may have any shape other than a circular shape and be configured to interfere with the extension tube 13 slightly. Changes in shape and configuration of the magnetic slide block shall be viewed as falling within the scope of the invention.

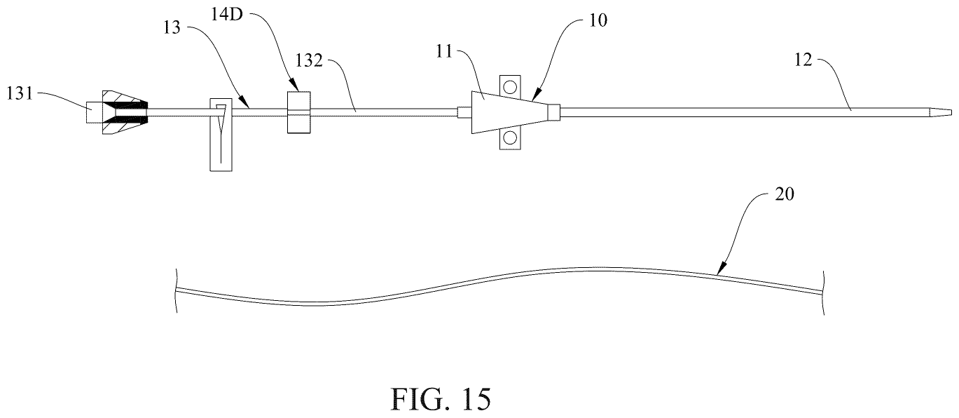

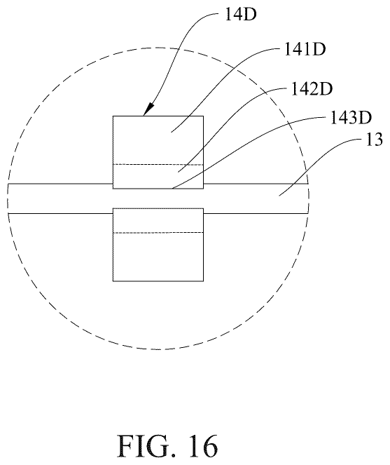

The fourth embodiment of the catheter capable of preventing guide wire retention according to the present invention is described below. Please refer to FIG. 15 and FIG. 16, which respectively show the exterior and a partial enlarged view of the fourth embodiment of the catheter capable of preventing guide wire retention according to the invention.

This embodiment is different from the first embodiment only in the design of the detachable magnetic attraction mechanism too; all the remaining parts of this embodiment are identical to their respective counterparts in the first embodiment and therefore will not be described repeatedly. In this embodiment, the detachable magnetic attraction mechanism includes a magnetic slide clip 14D provided on the tube body 132 of the extension tube 13. The magnetic slide clip 14D includes a clip element 141D and a magnetically attractive element 142D provided in the clip element 141D. The clip element 141D includes a positioning groove 143D for receiving the extension tube 13 to which the clip element 141D is clipped so that the clip element 141D can be moved on and along the extension tube 13 and removed from the extension tube 13. In one embodiment, the clip element 141D and the magnetically attractive element 142D of the magnetic slide clip 14D may jointly form a single magnet, or the clip element 141D may be implemented as a magnetic or non-magnetic element; all such changes shall be viewed as falling within the scope of the present invention. The positioning groove 143D of the magnetic slide clip 14D is designed according to the exterior shape of the extension tube 13, and the diameter of the positioning groove 143D is slightly smaller than the outer diameter of the extension tube 13 in order to provide a small amount of interference and thereby position the magnetic slide clip 14D on the extension tube 13. The positioning groove 143D of the magnetic slide clip 14D should be sized not to hinder the flow of liquid in the extension tube 13, but to provide a suitable clearance between the magnetic slide clip 14D and the extension tube 13 so that the magnetic slide clip 14D can be pushed along the extension tube 13. Like its counterpart in the third embodiment, the magnetic slide clip 14D is so designed that, once entering the extension tube 13, the guide wire 20 can be pushed out of the extension tube 13 through magnetic attraction by the magnetic slide clip 14D. As in the third embodiment, the guide wire 20 may be rendered magnetic either partially or by attaching an additional magnetic element to the guide wire 20.

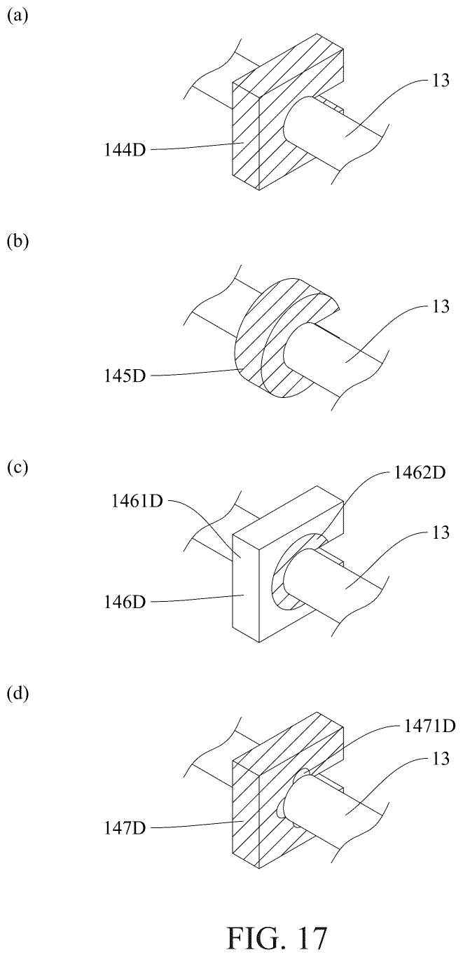

The shape of the magnetic slide clip 14D may be, for example but not limited to, any of the following. Please refer to FIG. 17, which shows various shapes and configurations of the magnetic slide clip in the fourth embodiment of the catheter capable of preventing guide wire retention according to the present invention. In the embodiment shown in FIG. 17(a), the magnetic slide clip 144D may have any shape, such as a square shape with an opening in one lateral side. In the embodiment shown in FIG. 17(b), the magnetic slide clip 145D may have any shape, such as a circular shape with an opening in one lateral side. In the embodiment shown in FIG. 17(c), the clip element 1461D of the magnetic slide clip 146D may be a non-magnetic housing of any shape, and the magnetically attractive element 1462D in the magnetic slide clip 146D may have any shape, such as an arc shape. In the embodiment shown in FIG. 17(d), the magnetic slide clip 147D may have any shape, and the positioning groove 1471D of the magnetic slide clip 147D may have any shape other than a circular shape and be configured to interfere with the extension tube 13 slightly. Changes in shape and configuration of the magnetic slide clip shall be viewed as falling within the scope of the invention.

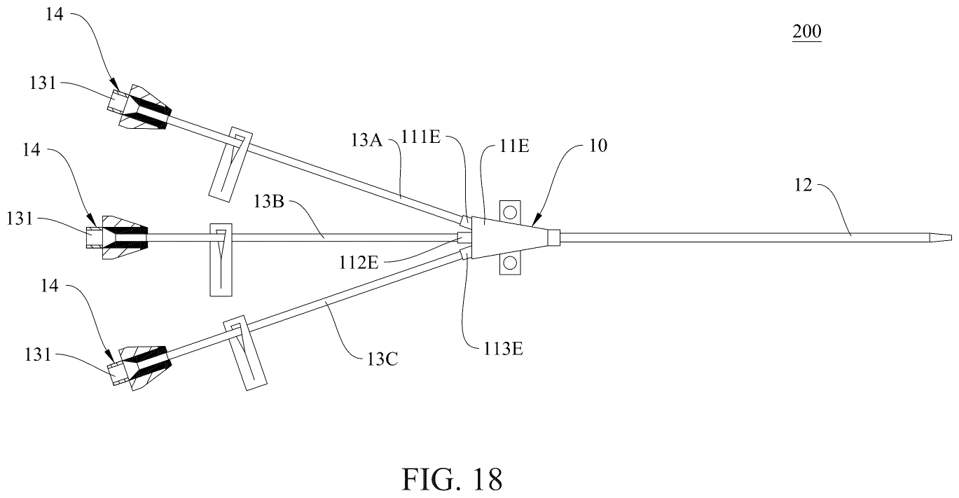

Please refer to FIG. 18, which shows the exterior of the fifth embodiment of the catheter capable of preventing guide wire retention according to the present invention. As shown in the drawing, the catheter 200 capable of preventing guide wire retention according to this embodiment may have a plurality of extension tubes (e.g., three extension tubes as disclosed in the drawing of this embodiment, namely the extension tubes 13A, 13B, and 13C), and the hub 11E may have a plurality of positioning holes (e.g., the positioning holes 111E, 112E, and 113E) corresponding in number to the extension tubes. By providing a plurality of extension tubes, different liquid medicines or nutrient solutions can be injected into a patient through the different extension tubes 13A, 13B, and 13C respectively. The invention has no limitation on the number of the extension tubes. Generally, the guide wire 20 is passed through the extension tube in the middle (i.e., the extension tube 13B), so it is feasible to provide the detachable magnetic attraction mechanism 14 at an opening of the middle extension tube 13B alone. Depending on practical needs, however, the detachable magnetic attraction mechanism 14 may be provided on any or each of the extension tubes instead; the invention has no limitation in this regard.

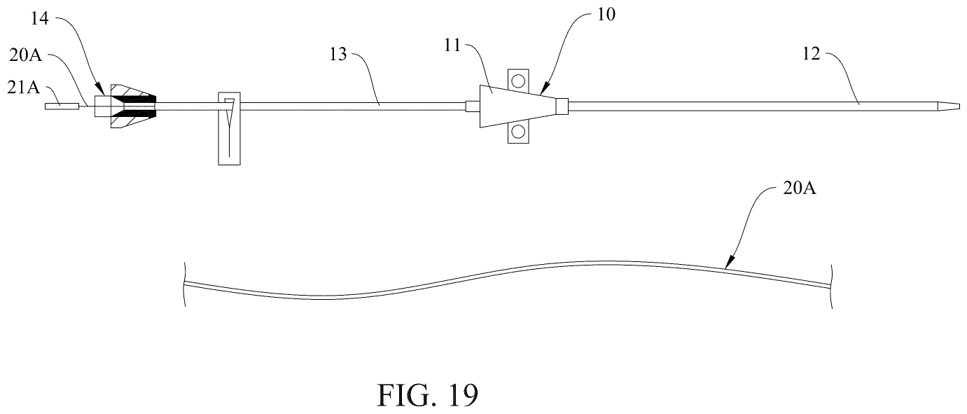

The guide wire 20 may be implemented in ways other than its foregoing embodiment. Please refer to FIG. 19, which shows a state of use of a different embodiment of the guide wire in the present invention. As shown in the drawing, the guide wire is not necessarily made entirely of a magnetic material. In the embodiment shown in FIG. 19, the guide wire 20A is made of a non-magnetic material but is provided with a magnetic element 21A. The magnetic element 21A can be attracted to the detachable magnetic attraction mechanism 14 so as to stabilize the guide wire 20A. The term “magnetic element” refers to a substance that can be attracted to a magnetically attractive material. More specifically, the magnetic element may be, for example, a substance that is composed of a transition element (e.g., iron, cobalt, or nickel) or an alloy thereof and that produces magnetism directly or indirectly; the invention has no limitation in this regard.

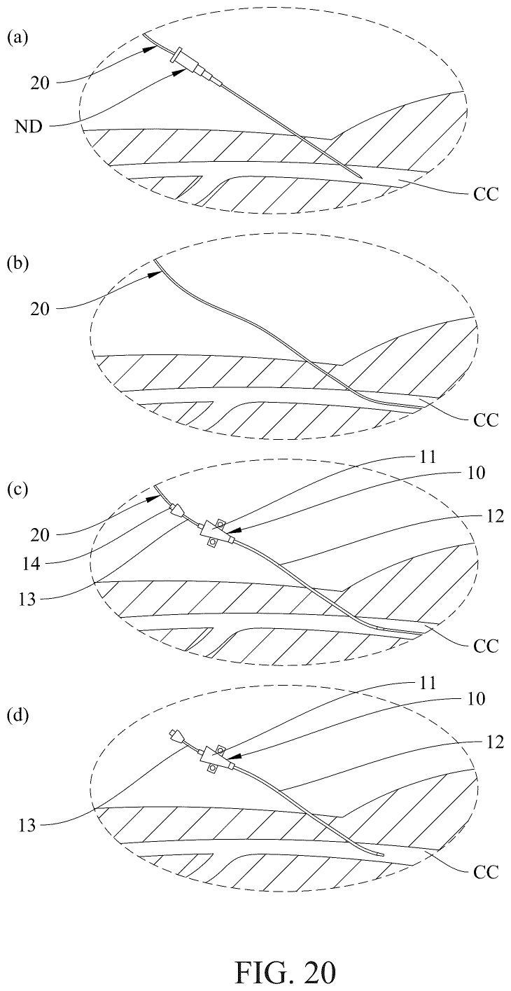

The operation of a venous catheter is illustrated in FIG. 20, which shows the process flow of guiding a central venous catheter according to the present invention into a vein. As shown in the drawing, a needle ND is inserted into the skin and enters a correct portion of a central vein CC, forming a hole through the skin that leads to the interior of the central vein CC. The guide wire 20 is then inserted through the needle ND into the central vein CC (see FIG. 20(a)). Following that, the needle ND is withdrawn, leaving the guide wire 20 in the wound (see FIG. 20(b)). Afterward, the catheter body 10 is passed over the guide wire 20, and the puncture needle 12 of the catheter body 10 is inserted into the skin along the guide wire 20 and is thus guided by the guide wire 20 into the correct portion of the central vein CC. During the process, the guide wire 20 is attracted to, and thus stabilized by, the detachable magnetic attraction mechanism 14 on the extension tube 13 (see FIG. 20(c)). Lastly, the guide wire 20 is withdrawn, leaving the catheter body 10 in the wound (see FIG. 20(d)), and the extension tube 13 of the catheter body 10 is connected to a liquid medicine or nutrient solution. With the detachable magnetic attraction mechanism 14 attracting and thereby stabilizing the guide wire 20 while the foregoing steps are performed, the guide wire 20 is kept from sliding into the catheter body 10 or even the patient's body.

In summary of the above, the present invention uses a detachable magnetic attraction mechanism to stabilize a guide wire, to ensure that the guide wire is exposed from one end of the catheter guided by the guide wire, and to thereby reduce the chance that the operator may forget to take out the guide wire. Moreover, the guide wire will be magnetically attracted to the detachable magnetic attraction mechanism and hence kept from sliding into a blood vessel inadvertently. The invention, therefore, can lower the risk of guide wire retention.

The above is the detailed description of the present invention. However, the above is merely the preferred embodiment of the present invention and cannot be the limitation to the implement scope of the invention, which means the variation and modification according to the present invention may still fall into the scope of the present invention.

What is claimed is:

1. A catheter capable of preventing guide wire retention, comprising:
a catheter body; and
a guide wire, wherein the guide wire is made of a magnetic material or provided with a magnetic element and is configured to be inserted through the catheter body;
wherein the catheter body is provided with at least one detachable magnetic attraction mechanism for attracting the guide wire and thereby holding the guide wire in position after the guide wire is inserted through the catheter body.

2. The catheter capable of preventing guide wire retention as claimed in claim 1, wherein the catheter body comprises a hub, a puncture needle provided on a side of the hub, and an extension tube connected to an opposite side of the hub; the catheter body further comprises a guide channel; the guide channel extends through the extension tube, the hub, and the puncture needle; and the guide wire is inserted into the puncture needle and then passed through the extension tube via the guide channel.

3. The catheter capable of preventing guide wire retention as claimed in claim 2, wherein the detachable magnetic attraction mechanism comprises a magnetic positioning member configured to be provided on an adapter of the extension tube, the magnetic positioning member comprises a base and a magnetically attractive element, the base is provided with a through hole, the base is further provided with a locking portion corresponding to an end of the through hole, the locking portion is configured to be locked to the adapter, and the magnetically attractive element is provided at the base.

4. The catheter capable of preventing guide wire retention as claimed in claim 2, wherein the detachable magnetic attraction mechanism comprises a magnetic suspension mechanism configured to be provided on the extension tube, and the magnetic suspension mechanism comprises a tie connected to the extension tube and a magnetically attractive element provided on the tie.

5. The catheter capable of preventing guide wire retention as claimed in claim 2, wherein the detachable magnetic attraction mechanism comprises a magnetic slide block provided on a tube body of the extension tube, the magnetic slide block comprises a slide base and a magnetically attractive element provided in the slide base, and the slide base comprises a positioning hole configured to allow passage of the extension tube.

6. The catheter capable of preventing guide wire retention as claimed in claim 2, wherein the detachable magnetic attraction mechanism comprises a magnetic slide clip provided on a tube body of the extension tube, the magnetic slide clip comprises a clip element and a magnetically attractive element provided in the clip element, and the clip element comprises a positioning groove for receiving the extension tube when the clip element is clipped to the extension tube.

7. A detachable magnetic attraction mechanism for stabilizing a guide wire, configured to be provided in relation to a catheter, the detachable magnetic attraction mechanism comprising a magnetic positioning member, the magnetic positioning member comprising:
a base, wherein the base is provided with a through hole, the base is further provided with a locking portion corresponding to an end of the through hole, and the locking portion is configured to be locked to an opening at an end of the catheter; and
a magnetically attractive element provided at the base and configured to attract the guide wire after the guide wire is inserted through the catheter.

8. A detachable magnetic attraction mechanism for stabilizing a guide wire, configured to be provided in relation to a catheter, the detachable magnetic attraction mechanism comprising a magnetic suspension mechanism, the magnetic suspension mechanism comprising:
a tie connected to the catheter; and
a magnetically attractive element provided on the tie and configured to attract the guide wire after the guide wire is inserted through the catheter, thereby exposing the guide wire from an opening at an end of the catheter.

9. A detachable magnetic attraction mechanism for stabilizing a guide wire, configured to be provided in relation to a catheter, the detachable magnetic attraction mechanism comprising a magnetic slide block, the magnetic slide block comprising:
a slide base comprising a positioning hole, wherein the positioning hole allows passage of the catheter; and
a magnetically attractive element provided in the slide base and configured to attract the guide wire after the guide wire is inserted through the catheter.

10. A detachable magnetic attraction mechanism for stabilizing a guide wire, configured to be provided in relation to a catheter, the detachable magnetic attraction mechanism comprising a magnetic slide clip, the magnetic slide clip comprising:
a clip element comprising a positioning groove, wherein the positioning groove is configured to receive the catheter when the clip element is clipped to the catheter; and
a magnetically attractive element provided in the clip element and configured to attract the guide wire after the guide wire is inserted through the catheter.
